(19) United States
(12) Patent Application Publication
(10) Pub. No.: US 2026/0061016 A1
(43) Pub. Date: Mar. 5, 2026

(54) Extrait aqueux des parties aériennes d'une plante du genre Aster et ses utilisations

(71) Applicants: TAUDERMA, CONTHEY (CH); INFLAMALPS, MONTHEY (CN)

(72) Inventors: Vincent MUTEL, ROLLE (CH); Julien HERITIER, SAVIESE (CH); Bruno SCHNYDER, CRANS MONTANA (CH); Xavier SIMONNET, FULLY (CH)

(21) Appl. No.: 18/846,802

(22) PCT Filed: Mar. 15, 2023
(86) PCT No.: PCT/EP2023/056558

§ 371 Date: Oct. 4, 2024

(30) Foreign Application Priority Data
Mar. 16, 2022 (FR) FR2202293

(51) Int. Cl. A61K 36/28; A61P 17/10

(52) U.S. Cl. CPC A61K 36/28; A61P 17/10; A61K 2236/15; A61K 2236/17; A61K 2236/331; A61K 2236/51

(57) ABSTRACT

The present invention relates to an aqueous extract obtained from a plant of the genus Aster for cosmetic or therapeutic applications, in particular in the prevention and/or treatment of acne.

### TECHNICAL FIELD

The invention relates to an aqueous extract obtained from a plant of the genus Aster for cosmetic or therapeutic applications, in particular in the prevention and/or treatment of acne.

### PRIOR ART

Acne is a common, chronic inflammatory skin disease of the pilosebaceous system. It affects most adolescents and accounts for more than half of all dermatology consultations. It is characterized by a hypersecretion of sebum (hyperseborrhea) leading to obstruction of the excretory duct of the pilosebaceous follicle. The obstruction then facilitates the proliferation of bacteria normally present in the skin, such as Cutibacterium acnes (C. acnes) or Staphylococcus aureus (S. aureus), responsible for inflammation and the appearance of red pimples. Depending on the severity of the condition, acne can cause physical complications as well as psychological discomfort.

The causes of acne are many and varied. Thus, genetic, hormonal and environmental factors, including smoking and pollution, promote inflammation through the production of cytokines. At the same time, dermal production of antimicrobial peptides and lipids controls skin bacteria, particularly C. acnes and S. aureus.

The commensal bacterium C. acnes, formerly known as Propionibacterium acnes and also including Propionibacterium avidum and Propionibacterium granulosum, is involved in maintaining healthy skin but can also act as an opportunistic pathogen in acne such as acne vulgaris, although Staphylococcus aureus (S. aureus) is also implicated in cutaneous acne.

To date, the majority of acne treatments have focused on reducing the overproduction of sebum due to hormonal imbalance, reducing the proliferation of C. acnes and S. aureus, and reducing the inflammatory consequences of these infections. However, most of these treatments target only one aspect of the etiology of acne. Moreover, C. acnes resistance to antibiotic treatments has gradually emerged over the years to become a worldwide problem, with high rates of 21% to 70% resistance reported for erythromycin (macrolides) and clindamycin (lincosamides), and less frequent resistance to tetracycline (4% to 30%).

The anti-inflammatory approach to acne, on the other hand, has focused mainly on general anti-inflammatory agents with low specificity and side effects.

There is therefore a need for a new approach to prevent and treat acne, while targeting all these etiologies and limiting the phenomenon of multi-resistant bacteria.

### SUMMARY OF THE INVENTION

To meet this need, the invention proposes a new aqueous extract obtained from the aerial parts of a plant of the genus Aster, in particular Aster alpinus and/or Aster tataricus.

It has recently been shown that C. acnes specifically triggers the release of IL-1β, a cytokine involved in the innate immune response (Li et al., Journal of Investigative Dermatology (2014) 134, 2747-2756).

However, the inventors have discovered that such an extract effectively combats IL-1β-stimulated IL-6 production by human epithelial cells, as well as inhibiting the proliferation of C. acnes and S. aureus.

These plants are mainly known in Mongolian and Chinese pharmacopoeia for oral administration in the treatment of fever and as cough medicine. In particular, extracts of these plants as used in pharmacopoeia are obtained from the root system and after alcoholic or hydroalcoholic extraction.

Thus, the invention relates to an aqueous extract obtained from the aerial parts of a plant of the genus Aster.

In particular, the plant can be selected from Aster alpinus, Aster tataricus, Aster nova-angliae, Aster umbellatus, Aster tongolensis, Aster radula, Aster novi-belgii, Aster macrophyllus, Aster laevis, Aster ericoides, Aster dumosus, Aster ageratoides, Aster frikantii, Aster cordifolius, Aster linosyris, Aster sedifolius, Aster laterifloris, Aster divaricatus, Aster amethystinus, Aster pyraneus, Aster pringlei, Aster sibiricus, Aster radula, Aster amellus and mixtures thereof.

Preferentially, the extract according to the invention is obtained from the aerial parts of Aster alpinus and/or Aster tataricus.

According to another object, the invention relates to the extract according to any of the preceding embodiments for use as a medication, in particular as an antibiotic and/or anti-inflammatory.

Preferentially, the extract is intended for use in the prevention and/or treatment of diseases associated with infection by C. acnes and/or S. aureus, including multi-resistant forms of C. acnes and/or S. aureus.

Infection with C. acnes and/or S. aureus is understood more preferentially as skin infections with C. acnes and/or S. aureus, but also to infections of prostheses with C. acnes and/or S. aureus.

Preferentially, the extract according to the invention is intended for use in the prevention and/or treatment of a disease selected from acne, psoriasis, vitiligo, acne vulgaris, seborrheic dermatitis and atopic dermatitis.

According to a preferred embodiment, the extract according to the invention is capable of being obtained by an extraction method comprising the following steps:
a. drying and grinding of aerial plant parts,
b. aqueous maceration of the aerial parts obtained in step a) at room temperature in the absence of organic solvents,
c. filtering the extract obtained in step b) and
d. concentrating the extract obtained in step c).

According to another object, the invention also relates to such a method for extracting the aqueous extract.

The invention also relates to a composition comprising the extract according to the invention. Thus, the composition can be pharmaceutical or cosmetic and comprises the extract according to the invention at a concentration greater than or equal to 0.1 mg/ml.

In the case of a cosmetic composition, it comprises the extract according to the invention and at least one cosmetically acceptable excipient. In the case of a pharmaceutical composition, it comprises the extract according to the invention and at least one pharmaceutically acceptable excipient.

Whatever the use, the composition is in a form suitable for topical application.

When the composition according to the invention is a pharmaceutical composition, it is intended for use as a medication.

When the composition according to the invention is a cosmetic composition, it is intended for use as a cosmetic product, that is, for treating healthy, non-diseased people.

Thus, the invention also relates to cosmetic uses of the extract or composition comprising it for its cosmetic effects, in particular for an anti-redness and/or anti-swelling effect or to combat oedema.

Other features and advantages will emerge from the detailed description of the invention, examples and FIGURES that follows.

### BRIEF DESCRIPTION OF THE FIGURES

FIG. 1 shows the anti-inflammatory effect on CACO-2 cells incubated with different extracts. The cells were stimulated with 0.25 ng/ml IL-1β, except where indicated “without IL-1β” (C0%), in triplicate. C100% are cells with no test sample. The resulting cell supernatants were diluted 1:10 directly after sampling and analyzed by ELISA for IL-6.

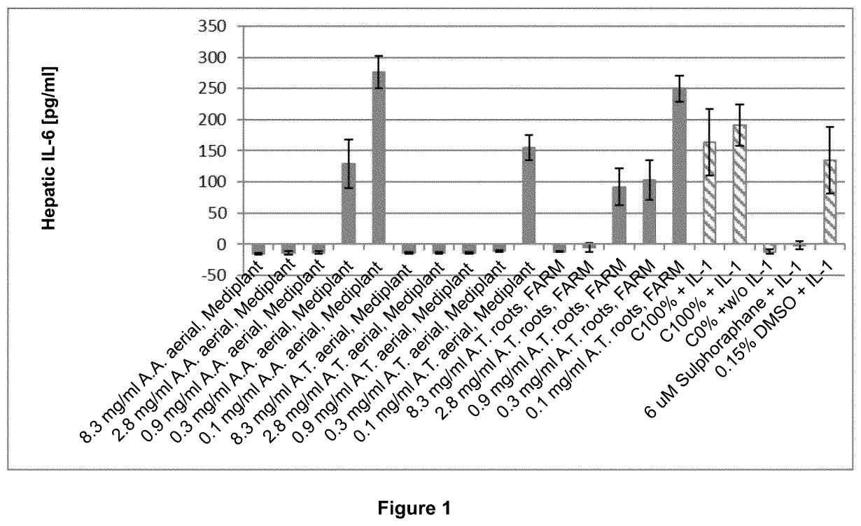

### DETAILED DESCRIPTION OF THE INVENTION

### Definition

By “extract” or “aqueous extract” of a plant of the genus Aster within the meaning of the invention, we mean a set of one or more molecules obtained from an extract of the genus Aster, preferentially from the aerial parts, according to an aqueous extraction method.

For the purposes of the invention, “aerial parts” refers to the vegetative apparatus of a plant, including organs such as the stem, buds, leaves, flowers and fruits, preferentially the leaves. By “aerial parts” we also mean all or part of the aerial parts, that is, the stem and/or the buds and/or the leaves and/or the flowers and/or possibly the fruit, preferentially the leaves.

By “treatment” in the sense of the invention, we mean the action of reversing, attenuating, inhibiting the progression of or preventing the disorder or condition to which this term applies. Preferentially, this means the action of reversing, attenuating, inhibiting the progression of or preventing diseases associated with infection by C. acne and/or S. aureus, more preferentially infections of prostheses by C. acne and/or S. aureus and/or acne, psoriasis, vitiligo, acne vulgaris, seborrheic dermatitis or atopic dermatitis.

For the purposes of the invention, “healthy subject” means a subject who may be a human being or an animal (other than a human being) not suffering from any pathology associated with infection by C. acne and/or S. aureus, such as infection of prostheses by C. acne and/or S. aureus, acne, psoriasis, vitiligo, acne vulgaris, seborrheic dermatitis or atopic dermatitis.

For the purposes of this invention, “medicinal product” means an active ingredient used for therapeutic purposes, whether alone or in combination with other active or non-active substances in medicinal formulations, including phytotherapy, intended for humans or animals suffering from a disease such as infection by C. acnes and/or S. aureus.

### Extract According to the Invention

The object of the present invention is therefore an aqueous extract obtained from a plant of the genus Aster, in particular an extract obtained from the aerial parts of the plant, more preferentially the leaves. Particularly preferably, the leaves are harvested and then dried before the extraction method.

Plants of the Aster genus are dicotyledons in the Asteraceae family, with many species. They are native to North America, Europe and the Himalayas. These are hardy perennials that flower in spring, summer or autumn, depending on the species. The best-known species are Aster alpinus or Alpine Aster, which blooms in early summer, and Aster tataricus or Tartarian Aster, which blooms in autumn.

Plants are made up of two distinct parts: the aerial part and the root part. With the exception of certain epiphytic plants, the root part is generally hidden underground, while the aerial part is visible.

The roots are divided into several parts. Sometimes they have a swollen part (rhizome, nodosities, etc.), sometimes they are smooth. The main roots are generally large and fleshy, and divide into smaller secondary roots and rootlets that capture nutrients. Roots have two functions: they anchor the plant in the soil, and they provide nourishment by absorbing water and mineral salts from the soil.

The aerial part groups together several organs: the stem, buds, leaves, flowers and possibly fruit.

In the context of the invention, the inventors are preferentially interested in the aerial parts of the plant, that is, all or part of the aerial parts (stem and/or buds and/or leaves and/or flowers and/or possibly fruits), in particular the leaves.

Thus, from all or part of the aerial parts, and in particular from the leaves, of plants of the Aster genus, the inventors have surprisingly identified that an aqueous extract makes it possible to obtain a biocidal effect, in particular on the bacteria responsible for acne, and an anti-inflammatory effect, in particular anti-IL-1β activity.

According to a particular embodiment, the extract according to the invention is obtained by aqueous maceration, preferentially at room temperature, in the absence of organic solvent.

Preferentially, the extract according to the invention may be obtained by a method comprising the following steps:
a. drying and grinding of aerial plant parts,
b. aqueous maceration of said aerial parts obtained in step a) at room temperature in the absence of organic solvent,
c. filtering the extract obtained in step b) and
d. concentrating the extract obtained in step c).

Optionally, the extraction method includes an additional drying step after extract concentration step d). In particular, drying can be carried out by freeze-drying. The extract according to the invention is then in the form of a powder or agglomerate.

Preferentially, extraction is carried out in the absence of organic, alcoholic or hydroalcoholic solvents. The use of such solvents makes it possible to extract toxic substances which are not desirable in the context of the invention. The extract according to the invention aims to be natural, healthy and non-toxic.

According to another particular embodiment, the extract according to the invention is obtained from a species selected from Aster alpinus, Aster tataricus, Aster nova-angliae, Aster umbellatus, Aster tongolensis, Aster radula, Aster novi-belgii, Aster macrophyllus, Aster laevis, Aster ericoides, Aster dumosus, Aster ageratoides, Aster frikantii, Aster cordifolius, Aster linosyris, Aster sedifolius, Aster laterifloris, Aster divaricatus, Aster amethystinus, Aster pyraneus, Aster pringlei, Aster sibiricus, Aster radula, Aster amellus and mixtures thereof.

Preferentially the species is Aster alpinus and/or Aster tataricus.

According to another object, the invention relates to the extract according to the invention for its use as a medication.

In one embodiment, the medication can be either an antibiotic, an anti-inflammatory medication, or an antibiotic and anti-inflammatory medication.

According to a variant, when the extract is used as an antibiotic and/or anti-inflammatory, the extract can be an aqueous extract obtained from a plant of the Aster genus, in particular an extract derived from the aerial parts and/or root system of a plant of the Aster genus.

When the extract is derived from the aerial parts and/or root system of a plant of the genus Aster, the Aster plant is preferentially selected from Aster alpinus, Aster tataricus, Aster nova-angliae, Aster umbellatus, Aster tongolensis, Aster radula, Aster novi-belgii, Aster macrophyllus, Aster laevis, Aster ericoides, Aster dumosus, Aster ageratoides, Aster frikantii, Aster cordifolius, Aster linosyris, Aster sedifolius, Aster laterifloris, Aster divaricatus, Aster amethystinus, Aster pyraneus, Aster pringlei, Aster sibiricus, Aster radula, Aster amellus and mixtures thereof, preferentially Aster alpinus and/or Aster tataricus.

According to a preferred embodiment, the extract according to the invention is used in the prevention and/or treatment of diseases associated with infection by C. acnes and/or S. aureus, including multi-resistant forms of C. acnes and S. aureus.

Diseases associated with C. acnes and/or S. aureus infection are preferentially selected from C. acnes and/or S. aureus skin infections and C. acnes and/or S. aureus prosthesis infections.

In the case of skin infection by C. acnes and/or S. aureus, the disease is preferentially selected from acne, psoriasis, vitiligo, acne vulgaris, seborrheic dermatitis and atopic dermatitis.

Prosthesis infection, also known as periprosthetic infection or prosthetic infection, is a rare but dreaded complication, as its precise diagnosis is complex.

Its management therefore requires collaboration between an orthopedic physician and an infectiologist. Ignorance of the diagnosis can have serious repercussions, including removal of the prosthesis or even sepsis. Identifying the germ responsible for the infection is therefore essential. Infections associated with prosthesis surgery are frequently linked to C. acnes and/or S. aureus (>31%).

When there is a suspicion of bacterial infection following implantation of a prosthesis, it is in our interest to treat it as quickly as possible. This type of infection can be acute or chronic. Acute infection is rapid and can lead to septicemia. However, treatment is fairly well managed with conventional antibiotics.

Chronic infections are more complex, reveal themselves later and are less accessible to conventional antibiotics. In fact, a biofilm is produced by the bacteria responsible for infection, such as C. acnes, making accessibility to antibiotics difficult. The production of this biofilm is notably induced by the production of II-1β. It is therefore also of interest for preventing and/or treating prosthesis infections caused by C. acnes and/or S. aureus.

### Composition According to the Invention

According to another object, the invention concerns a composition comprising the extract according to the invention, and an ingredient chosen from glycerol or glycerine, alcohol, oil and water. By way of example, for a liquid formulation, the aqueous extract is mixed with glycerine in proportions of between 50 and 80% by weight of glycerine, in order to be microbiologically stabilized.

When the extract according to the invention is in dry form, it can be resuspended in a solution chosen from glycerol, alcohol, oil or water.

The composition according to the invention may be in the form of an aqueous composition, an oily composition, an emulsion or an aqueous bead.

The composition according to the invention can be in a form suitable for topical application, preferentially in a form chosen from a cream, serum, mask, balm and lotion.

The extract according to the invention included in the composition may be present in a concentration of at least 0.1 mg/ml.

Preferentially, the concentration is less than or equal to 0.9 mg/ml. More preferentially, the concentration is between 0.1 mg/ml and 0.9 mg/ml.

The composition according to the invention, in addition to the extract according to the invention, may comprise other constituents such as one or more other active ingredient(s) such as, for example, plant extracts comprising antioxidants and/or hyaluronic acid and/or collagen.

According to a preferred embodiment, the composition according to the invention comprises at least one pharmaceutically acceptable excipient, that is, an ingredient which is useful for the preparation of a pharmaceutical composition, and which is safe and non-toxic, for pharmaceutical use.

When the composition according to the invention comprises a pharmaceutically acceptable excipient, it is intended for use as a medication.

According to another embodiment, the composition according to the invention comprises at least one cosmetically acceptable excipient, that is to say which does not cause sensations of discomfort for the user, such as redness, tightness or tingling.

Cosmetically acceptable excipients may be selected from oils, such as silicone oils, linear or cyclic, volatile or non-volatile, or vegetable oils; waxes, such as ozokerite, polyethylene wax, beeswax or carnauba wax, or vegetable waxes; silicone elastomers; surfactants, preferably emulsifiers, whether non-ionic, anionic, cationic or amphoteric; co-surfactants, such as linear fatty alcohols; thickeners and/or gelling agents; humectants, such as polyols like glycerine; colorants, preservatives, fillers; tensors; sequestrants; fragrances; hyaluronic acid; collagen, plant extracts or floral waters.

When the composition according to the invention comprises a cosmetically acceptable excipient, it is intended for use as a non-therapeutic cosmetic product for healthy, non-diseased subjects.

### Method

The extract can be obtained by any aqueous extraction method known to the person skilled in the art.

In particular, the extraction method may comprise the following steps:
a. drying and grinding of aerial plant parts,
b. aqueous maceration of the aerial parts obtained in step a) at room temperature in the absence of organic solvents,
c. filtering the extract obtained in step b) and
d. concentrating the extract obtained in step c).

Advantageously, the method can be carried out at room temperature. Preferentially, the method does not include an extraction step involving the addition of an alcoholic or hydroalcoholic solution.

The extract is then obtained in liquid form. When the extract is in solid form, the method also includes a freeze-drying step.

Thus, the extract may be in solid form or in liquid form.

### Cosmetic Use

According to another object, the invention concerns a cosmetic use of an extract according to the invention or of a composition according to the invention for a cosmetic effect, in particular an anti-redness and/or anti-swelling effect and/or to combat oedema.

Cosmetic effects are chosen in particular from anti-redness, anti-swelling, purifying, cleansing, combating edema, reducing skin imperfections, improving skin quality, reducing comedones, reducing blackheads and reducing oily skin.

The invention is now illustrated by non-limiting examples of compositions according to the invention and by results.

### EXAMPLES

### Example 1—Aster alpinus Extract According to the Invention

The extract is obtained by the extraction method described below. The method comprises the following steps:
a. drying and grinding of aerial plant parts,
b. aqueous maceration of the aerial parts obtained in step a) at room temperature in the absence of organic solvents,
c. filtering the extract obtained in step b) and
d. concentrating the extract obtained in step c).

The extract obtained is then in the form of an aqueous extract containing, in particular, sugars and flavonoids.

### Example 2—Aster tataricus Extract According to the Invention

The extract is obtained by the method described in Example 1. The extract obtained is then in the form of an aqueous extract containing, in particular, sugars and flavonoids.

### Example 3—Extract According to the Invention in Solid Form

The extract is obtained according to the method described in Example 1 or 2 and includes a final additional freeze-drying step to obtain either an extract of Aster alpinus in solid form, or an extract of Aster tataricus in solid form.

### Example 3

### Test of the Antibacterial Effect of an Extract According to the Invention and without the Invention

The aim of the test is to compare the efficacy of an extract according to Example 1 or Example 2 and an extract of the Aster genus obtained from the root system (Without the Invention). The aim of the test is to identify the minimum inhibitory concentration (MIC) on a strain of S. aureus registered under number DSM799.

The minimum inhibitory concentration (MIC) was determined in vitro using S. aureus solution culture in 96-well plates. The initial concentration of S. aureus was 1.25*10\hat{ }5 CFU/ml. After 48 h incubation in the presence of a series of dilutions of the extract, a 20 μl drop was taken and placed on a TSB agar plate. The diameter of the “drop culture” is about 1.5 cm. Agar plate cultures were then incubated at 37° C. for 14 hours.

The positive control corresponds to the maximum growth of a bacterium, that is, when it is fully confluent, meaning that bacterial colonies stick together and none are visible. Different extracts according to the invention and not according to the invention at different concentrations are thus compared. When no colonies, or separate individual colonies, are visible, then the bacteria have been inhibited by the extract. The extract concentration is then defined as MIC.

### Results

Extracts in solid form according to Example 3 were used for this test. Extracts were diluted by one-third. For each extract, the following concentrations were tested: 8.3 mg/ml, 2.8 mg/ml, 0.9 mg/ml, 0.3 mg/ml and 0.1 mg/ml.

The results are presented in Table 1 below.

TABLE 1

| | MIC | Number of |
|---|---|---|
| | [mg/ml] | colonies |
| Chloramphenicol | 0.05 | 0 |
| 1) Aster alpinus - aerial parts, | 0.9 | 0 |
| (Example 1) | | |
| 2) Aster alpinus - roots, | 2.8 | 0 |
| (Without the Invention) | | |
| 3) Aster tataricus - aerial parts, | 0.9 | 0 |
| (Example 2) | | |
| 4) Aster tataricus - roots, | 8.3 | 0 |
| (Without the Invention) | | |

The figures in the table (in mg/ml) indicate Minimum Inhibitory Concentrations (MIC). The definition of MIC is as follows: Arbitrary units of bacterial growth were determined on agar culture plates (drop method). Cultures where the number of colonies was reduced from confluence (maximum growth) to observable single colonies were defined as MICs.

The results show that Aster alpinus extract obtained from the aerial parts and Aster tataricus extract obtained from the aerial parts have a strong inhibitory effect on S. aureus. Conversely, extracts obtained from the roots of Aster alpinus and Aster tataricus have a weak effect. The MIC was 0.9 mg/ml for both extracts when tested on S. aureus and 0.3 mg/ml for both extracts when tested on C. acnes. Consequently, the origin of the plant is an essential characteristic of the extract to obtain such an effect.

### Test of the Anti-Inflammatory Effect of an Extract According to the Invention and without the Invention

The aim of the test is to compare the efficacy of an extract according to Example 1 or Example 2 and an extract of the Aster genus obtained from the root system (Without the Invention). The trial aims to evaluate the anti-inflammatory effect of IL-1β on human Caco-2 endothelial cells.

Extracts according to the invention in solid form were used for this test. Extracts in solid form were dissolved in a medium (containing penicillin and streptomycin) using a vortex, and incubated for approximately 1 hour at 37° C. A series of 1/3 dilutions was then prepared. For each extract, the following concentrations were tested: 8.3 mg/ml, 2.8 mg/ml, 0.9 mg/ml, 0.3 mg/ml and 0.1 mg/ml. Caco-2 cells (P60) were at 60% confluence at the start of the test. After 24 h incubation of the cells with the extracts, 80 μl had been transferred to an intermediate fresh 96-well plate and centrifuged. The supernatant was then diluted 1:10 on the second intermediate plate, then transferred to the coated ELISA plate to measure IL-6 content.

### Results

The aerial parts of Aster alpinus and Aster tataricus inhibit IL-1β-stimulated IL-6 secretion in a concentration-dependent manner, with an IC50 of 0.2 mg/ml for both extracts. For Aster tataricus roots, the IC50 was higher, close to 0.9 mg/ml, indicating that the root extract is less active than the extract of aerial parts. Concentrations that inhibited 50% of IL-1β-induced IL-6 production were 0.14 mg/ml for Aster tataricus extract and 0.3 mg/ml for Aster alpinus extract.

Thus, extracts obtained from aerial parts have an anti-inflammatory effect.

1. An aqueous extract obtained from a plant of the species Aster alpinus and/or Aster tataricus, characterized in that it is an extract obtained from the aerial parts of the plant.

2. The extract according to claim 1, characterized in that the aerial parts are the leaves.

3. The extract according to claim 1, characterized in that the extract is obtained by aqueous maceration at room temperature.

4. The extract according to claim 1, obtainable by the extraction method comprising the following steps:
a. drying and grinding of aerial plant parts,
b. aqueous maceration of the aerial parts obtained in step a) at room temperature in the absence of organic solvents,
c. filtering the extract obtained in step b) and
d. concentrating the extract obtained in step c).

5. The extract according to claim 4, characterized in that the extraction is carried out in the absence of alcoholic or hydroalcoholic solvent.

6. The extract according to claim 1, for use thereof as a medication.

7. The extract for use according to claim 6, as antibiotic and/or anti-inflammatory.

8. The extract for use according to claim 6, in the prevention and/or treatment of diseases associated with infection by C. acnes and/or S. aureus.

9. The extract for use according to claim 6, wherein the diseases associated with C. acne and/or S. aureus infection are selected from C. acnes and/or S. aureus skin infections and C. acnes and/or S. aureus prosthesis infections.

10. The extract for use according to claim 6, in the prevention and/or treatment of a disease selected from acne, psoriasis, vitiligo, acne vulgaris, seborrheic dermatitis and atopic dermatitis.

11. An aqueous extract obtained from a plant of the genus Aster for use in the prevention and/or treatment of diseases associated with infection by C. acnes and or S. aureus.

12. The extract for use according to claim 11, characterized in that the aqueous extract is obtained from a species selected from Aster alpinus, Aster tataricus, Aster nova-angliae, Aster umbellatus, Aster tongolensis, Aster radula, Aster novi-belgii, Aster macrophyllus, Aster laevis, Aster ericoides, Aster dumosus, Aster ageratoides, Aster frikantii, Aster cordifolius, Aster linosyris, Aster sedifolius, Aster laterifloris, Aster divaricatus, Aster amethystinus, Aster pyraneus, Aster pringlei, Aster sibiricus, Aster radula, Aster amellus and mixtures thereof.

13. A composition comprising the extract according to claim 1 and glycerol and alcohol and water.

14. The composition according to claim 13, characterized in that the concentrating the extract is at least 0.1 mg/ml.

15. The composition according to claim 13 also comprising at least one pharmaceutically acceptable excipient or at least one cosmetically acceptable excipient.

16. The composition according to claim 15, in a form suitable for topical application.

17. The composition according to claim 13, characterized in that it is in a form chosen from a cream, a serum, a mask and a lotion.

18. The composition according to claim 13, for use thereof as a medication.
